SEQUENCE LISTING (8 sequences)

SEQ ID NO: 1 - protein, 16 aa, synthetic construct
TGLDKGNHEGEDLGYQ

SEQ ID NO: 2 - protein, 25 aa, synthetic construct
YGRKKRRQRRRVELKKFKLKLLLSV

SEQ ID NO: 3 - protein, 8 aa, synthetic construct
KLGLKGYY

SEQ ID NO: 4 - protein, 14 aa, synthetic construct
NSDVDARRLLEATL

SEQ ID NO: 5 - protein, 13 aa, synthetic construct
RPFERDISNVPFS

SEQ ID NO: 6 - protein, 10 aa, synthetic construct
IPGPTFLDPH

SEQ ID NO: 7 - protein, 30 aa, synthetic construct
FQGTFPDGFLWAVGSAAYQTEGGWQQHGKG

SEQ ID NO: 8 - protein, 11 aa, synthetic construct
PEQLERALNSS

(19) United States
(12) Patent Application Publication
(10) Pub. No.: US 2026/0226112 A1
(43) Pub. Date: Aug. 6, 2026

(54) SYSTEMS AND METHODS FOR POST-VACCINE SPIKE NEUTRALIZATION MULTIVALENT PEPTIDE DETECTION AND ENGINEERING

(71) Applicant: Neo7Bioscience, Inc., Dallas, TX (US)

(72) Inventors: John A. Catanzaro, Rockwall, TX (US); Philipp Anokhin, Washington, DC (US); Natalia Von Ranke, Rio de Janeiro (BR); Wei Zhang, Houston, TX (US)

(21) Appl. No.: 19/463,419

(22) Filed: Jan. 29, 2026

Related U.S. Application Data
(60) Provisional application No. 63/752,141, filed on Jan. 31, 2025.

(51) Int. Cl. C07K 14/005; G06N 20/00

(52) U.S. Cl. CPC C07K 14/005; G16B 40/00

(57) ABSTRACT

Systems, methods, and compositions involving post-vaccine spike neutralization multivalent peptide detection and engineering are described herein. In an embodiment, a system for post-vaccine spike neutralization multivalent peptide detection and engineering may include at least a processor and a memory communicatively connected to the at least a processor, wherein the memory contains instructions configuring the at least a processor to: receive transcriptomic data, analyze the transcriptomic data using bioinformatic surveillance, modeling, ranking, mapping, and confidence selection, and output a personalized spike neutralization multivalent peptide sequence as a function of the transcriptomic data.

### CROSS-REFERENCE TO RELATED APPLICATIONS

This application is a Non-provisional application of U.S. Provisional Application No. 63/752,141 filed on Jan. 31, 2025, and entitled “SYSTEMS AND METHODS FOR POST-VACCINE SPIKE NEUTRALIZATION MULTIVALENT PEPTIDE DETECTION AND ENGINEERING” the entirety of which is incorporated by reference in its entirety.

### FIELD OF THE INVENTION

The present invention generally relates to the field of immunology. In particular, the present invention is directed to systems and methods for post-vaccine spike neutralization multivalent peptide detection and engineering.

### REFERENCE TO ELECTRONIC SEQUENCE LISTING

The present application includes a Sequence Listing in ST.26 format as an XML file entitled “35877-100401_SL”, which was created on Jan. 28, 2026 and which has a size of 8,039 bytes. The contents of XML file “35877-100401_SL” are incorporated by reference herein in its entirety. The contents of the electronic sequence listing entitled “1399-003USP1.xml” having the following size 7,587 bytes, which was created Jan. 17, 2025, is incorporated by reference in its entirety.

### SUMMARY OF THE DISCLOSURE

In an aspect, a system for post-vaccine spike neutralization multivalent peptide detection and engineering, wherein the system comprises at least a processor; and a memory communicatively connected to the at least a processor, wherein the memory contains instructions configuring the at least a processor to receive transcriptomic data; analyze the transcriptomic data using bioinformatic surveillance, modeling, ranking, mapping, and confidence selection; output a personalized spike neutralization multivalent peptide sequence as a function of the transcriptomic data.

In yet another non-limiting aspect, a method for post-vaccine spike neutralization multivalent peptide detection and engineering, wherein the method comprises obtaining a blood sample; performing high-definition RNA transcriptomics, wherein performing high-definition RNA transcriptomics produces transcriptomic data; analyzing the transcriptomic data using bioinformatic surveillance, modeling, ranking, mapping, and confidence selection; and outputting a personalized spike neutralization multivalent peptide sequence as a function of the transcriptomic data.

In yet another non-limiting aspect, a post-vaccine spike neutralization multivalent peptide sequence, wherein the post-vaccine spike neutralization multivalent peptide sequence comprises anti-spike peptide; NLRP3 mitigation: CD40 mitigation; IL-mitigation; IL-10 potentiation; anti-galectin-3; KL-FG_RXKF; and EP-PEQL.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is an exemplary block diagram for a system for post-vaccine spike neutralization multivalent peptide detection and engineering;

FIG. 2 is a block diagram of an exemplary machine-learning process;

FIG. 3 is a diagram of an exemplary embodiment of a neural network;

FIG. 4 is a diagram of an exemplary embodiment of a node of a neural network;

FIG. 5 is a flow diagram of an exemplary method for post-vaccine spike neutralization multivalent peptide detection and engineering; and

FIG. 6 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted. The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems, methods, and compositions for post-vaccine spike neutralization multivalent peptide detection and engineering. In an embodiment, the present disclosure may provide for a pooled peptide formulation developed to address the side effects of RNA-based vaccines.

Aspects of the present disclosure can be used to detect and address molecular disruptions caused by viral infections and mRNA vaccines. This may be accomplished by focusing on RNA transcription, protein integrity, and immune system recalibration. Aspects of the present disclosure can also be used to engineer personalized treatment strategies. This is so, at least in part, because the present systems and methods allow for the detection and engineering of multivalent pooled-peptide formulations.

Aspects of the present disclosure allow for detection and engineering of post-vaccine spike neutralization multivalent peptides. Exemplary embodiments illustrating aspects of the present disclosure are described below in the context of several specific examples.

Exemplary embodiments of a system for post-vaccine spike neutralization multivalent peptide detection and engineering are described herein. In an embodiment, system for post-vaccine spike neutralization multivalent peptide detection and engineering may include at least a processor and a memory communicatively connected to the at least a processor, wherein the memory contains instructions configuring the at least a processor to receive transcriptomic data, analyze the transcriptomic data using bioinformatic surveillance, modeling, ranking, mapping, and/or confidence selection, and outputting a personalized spike neutralization multivalent peptide sequence as a function of the transcriptomic data.

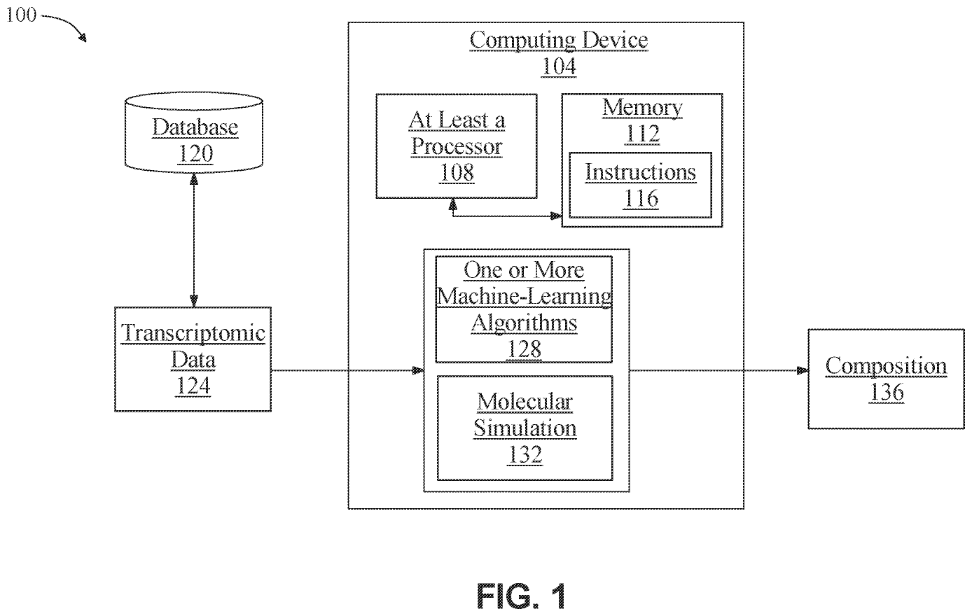

Now referring to FIG. 1, a system 100 for post-vaccine spike neutralization multivalent peptide detection and engineering is illustrated. In an embodiment, system 100 may include at least a processor 108 and a memory 112 communicatively connected to the at least a processor 108, wherein the memory 112 contains instructions 116 configuring the at least a processor 108 to receive transcriptomic data 124, analyze the transcriptomic data 124 using bioinformatic surveillance, modeling, ranking, mapping, and confidence selection, and output a personalized spike neutralization multivalent peptide sequence as a function of the transcriptomic data 124.

In continued reference to FIG. 1, system for post-vaccine spike neutralization multivalent peptide detection and engineering may include a computing device 104. Computing device 104 may include any computing device as described in this disclosure, including without limitation a microcontroller, microprocessor, digital signal processor (DSP) and/or system on a chip (SoC) as described in this disclosure. Computing device 104 may include, be included in, and/or communicate with a mobile device such as a mobile telephone or smartphone. Computing device 104 may include a single computing device operating independently or may include two or more computing devices operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device, which may operate in parallel, in series, redundantly, or in any other manner used for distribution of tasks or memory between computing devices. Computing device 104 may be implemented using a “shared nothing” architecture in which data is cached at the worker, in an embodiment, this may enable scalability of system for post-vaccine spike neutralization multivalent peptide detection and engineering and/or computing device 104.

Computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

In an embodiment, system 100 may include HD-Spike X Detect, which may include a molecular surveillance panel that offers an approach to understanding and addressing the molecular disruptions caused by viral infections and mRNA vaccines. In an embodiment, HD-Spike X Detect may include molecular surveillance panels specifically tailored for genetic safety surveillance. For purposes of this disclosure, “genetic safety surveillance” refers to the ongoing monitoring and evaluation of genetic information, practices, and technologies to ensure they are used safely and ethically. This may, for example, include tracking potential risks associated with genetic modifications, gene therapies, and/or genetic testing. These insights may be provided by focusing on RNA transcription, protein integrity, and immune system recalibration. Further, these insights may provide healthcare providers with precise insights for developing personalized treatment strategies.

RNA transcription detection may focus on identifying active RNA linked to ongoing spike protein production and/or other aberrant proteins. This may be accomplished by detecting lingering molecular activity after infections, including SARS-CoV-2 and/or mRNA vaccines. This may unveil hidden triggers of chronic symptoms, such as fatigue, brain fog, and/or systemic inflammation.

Protein integrity monitoring may focus on monitoring functional protein loss that impairs cellular and metabolic processes. This may be accomplished by detecting abnormal protein expressions affecting tissue repair and/or immune responses. This may be useful in identifying protein imbalances contributing to progressive health decline and chronic diseases.

Genetic integrity checks may focus on evaluating whether viral and/or vaccine-related RNA has integrated into the DNA exome. This may be accomplished by monitoring genetic instability leading to protein malfunctions and/or immune dysregulation. This may help uncover hidden molecular disturbances linked to chronic illness and/or cellular dysfunction.

Immune system recalibration assessments may focus on detecting disruptions in HLA gene activity, critical for immune system regulation. It may identify markers of immune confusion, chronic inflammation, and/or autoimmune triggers. This may allow for restoration in immune balance preventing uncontrolled inflammation and immune misfires.

In an embodiment, the Spike X Detect panel may address molecular-level disturbances that conventional diagnostics may miss, offering proactive solutions to prevent long-term health complications. For example, and without limitation, Spike X Detect panel may assist in stopping destructive hyperinflammation, provide vascular health protection, build neurological resilience, assist in immune system regulation, and help restore protein integrity. This may be so because Spike X Detect panel may provide early detection of inflammatory pathways to prevent damage to tissues and organs, lower the risk of clotting, cardiovascular events, and/or vascular injury from molecular stress, mitigate neuroinflammation to protect against cognitive decline, brain fog, and/or neurodegenerative disorders, prevent autoimmune responses by recalibrating immune recognition and/or self-regulation, and/or address functional protein loss to optimize metabolism, cellular repair, and/or organ function.

In an embodiment, the panel may also identify molecular triggers that may activate cancer-related pathways, including aberrant RNA and/or protein signaling. This insight may be crucial for individuals at higher risk of cancer due to viral infections, vaccine-related disruptions, and/or genetic instability, allowing for early intervention.

In an embodiment, Spike X Detect panel may provide peptide-based mitigation strategies. For example these strategies may include neutralizing spike proteins and/or other aberrant molecules lingering in tissues and circulation, rebalancing HLS gene activity to reduce chronic inflammation and/or immune confusion, protecting cardiovascular health by mitigating clotting risks, supporting cognitive function and/or neurological recovery by addressing brain inflammation, and/or repairing function al proteins to optimize metabolism, tissue regeneration, and/or cellular performance.

In an embodiment, a specimen needed for the Spike X Detect panel may include whole blood (10 mL) for comprehensive RNA, protein, and/or genomic analysis. The methodology in such an analysis may include high-definition RNA transcriptomics and/or bioinformatic surveillance, modeling, ranking, mapping, and/or confidence selection. Further, in some embodiments, the above methodologies may utilize machine-learning, large-language models, and/or molecular simulation 132. Overall, the turnaround time for the Spike X Detect panel may take about 4-5 weeks following sample receipt. The Spike X Detect panel may include other blood based samples, including but not limited to serum, plasma, buffy coat, dried blood spots, and peripheral blood mononuclear cells (PBMCs).

With continued reference to FIG. 1, the Spike detect panel may include other biological samples, including but not limited to buccal swabs, saliva, hair follicles, skin biopsies, bone marrow aspirates, cord blood, midstream urine, catheterized urine, 24-hour urine collection, urinary sediment, cerebrospinal fluid (CSF), synovial fluid, pleural fluid, peritoneal fluid, ascitic fluid, pericardial fluid, semen, vaginal secretions, amniotic fluids, breast milk, nasal swabs, nasopharyngeal swabs, oropharyngeal swabs, sputum, bronchoalveolar lavage (BAL), skin biopsy, liver biopsy, kidney biopsy, lung biopsy, muscle biopsy, lymph node biopsy, endometrial biopsy, gastrointestinal biopsies, stool samples, sweat samples, hair sample, nail sample, and the like.

In continued reference to FIG. 1, “molecular simulation,” as described herein, refers to the use of computational methods to model and study the behavior, interactions, and properties of molecules and materials. Molecular simulation 132 may allow scientists and researchers to gain insights into the structure and dynamics of molecular systems without the need for physical experiments. Molecular simulation may include any molecular modeling and organoid modeling as described in U.S. Nonprovisional patent application Ser. No. 19/455,085 filed on Jan. 21, 2026 and entitled “APPARATUS AND METHOD FOR BIDIRECTIONAL DATA INTEGRATION” which is incorporated by reference in its entirety. For example, molecular simulation 132 may aid in predicting how molecules interact, how they fold, and/or how they behave under different conditions, such as temperature, pressure, and/or solvent environments. In an embodiment, molecular simulation 132 may include molecular dynamics (MD) simulations, Monte Carlo (MC) simulations, quantum mechanical simulations, course-grained simulations, and/or the like. MD simulations may simulate the time evolution of a system by solving Newton's equations of motion for atoms and/or molecules. MD simulations may be used to understand the dynamics of molecules over time. MC simulations are a statistical method that uses random sampling to explore the configuration space of a system. MC simulations may be used for systems that require large-scale sampling of possible configurations, especially for equilibrium properties. Quantum mechanical simulations may focus on the behavior of atoms and elections using quantum mechanics. For example, quantum mechanical simulations may include methods such as density functional theory (DFT) and/or Hartree-Fock. Course-grained simulations may group atoms and represent the group of atoms by a single particle to simplify complex systems and allow simulations of larger systems over longer timescales.

With further reference to FIG. 1, in an embodiment, running a molecular simulation 132 may involve solving equations of motion, energy calculations, and/or statistical methods to explore how molecules behave over time and/or under different conditions. In a non-limiting embodiment, running a molecular simulation 132 may include system setup, wherein system setup may include defining the system and selecting the force field, choosing a simulation method, running the simulation, monitoring the simulation, and analyzing the results. In some cases, the results of the molecular simulation 132 may be visualized. For example, the results of the molecular simulation 132 may be visualized in 3D to better understand the molecular behavior. This may be accomplished using specialized software, such as VMD, Chimera, and/or the like, to create animations of molecular dynamics and/or to analyze the structure of molecules. Further, in some cases, running a molecular simulation 132 may involve optimization and/or validation through convergence and comparisons with experimental data.

Still referring to FIG. 1, in an embodiment, system setup may include defining the system of interest. For example, this may include specifying the types of molecules, their atomic positions, and in some cases, the environment. Additionally, system setup may include selecting the force field. As used herein “force field” is a set of mathematical functions used to model the interatomic interactions between atoms and molecules. The force field may define how atoms interact with each other such as bond lengths, angles, van der Waals forces, and/or electrostatic interactions. In an embodiment, force field may include Chemistry at Harvard Macromolecular Mechanics (CHARMM), Assisted Model Building with Energy Refinement (AMBER), and/or Optimized Potentials for Liquid Simulations (OPLS). Further, in some cases system setup may include defining boundary conditions. Molecular systems may be computationally expensive to simulate at full size. To combat this, periodic boundary conditions (PBC) may be used, where the system is repeated in space, creating the illusion of an infinite system. For example, a small box of molecules may be simulated as if it were part of a larger, repeating system.

With continued reference to FIG. 1, in an embodiment, a simulation method may be chosen, which may include any one of the simulation methods as previously described. In an embodiment, once a simulation method is selected, the simulation may be run. Running the simulation may include energy calculations, force calculations, and/or updating positions and velocities as a function of the energy and/or force calculations. At each step, a system's total energy may be computed based on interactions between atoms. This energy may be composed of bond energy, angle bending, Van der Waals forces, and/or electrostatic interactions. Force calculations may include forces acting on each atom derived from the energy function. These forces may determine how atoms move during the simulation. In an embodiment implementing MD simulations, once forces are calculated, the positions and velocities of atoms may be updated according to the equations of motion. In some cases, this may be calculated using numerical methods such as the Verlet algorithm. Alternatively, in embodiments implementing MC simulations, random moves may be made, and the energy is recalculated to determine if the new configuration is accepted. As the simulation progresses, several properties of interest may be monitored. For example, this may include temperature, pressure, energy, and conformations (how molecules and/or structures change over time).

Further referencing FIG. 1, in an embodiment, analyzing the results of the molecule simulation may include applying trajectory analysis, radial distribution function (RDF), root mean square deviation (RMSD), and/or free energy calculations to the results. Trajectory analysis may include visualizing the path of each molecule and/or atom and examining how the system evolves over time. RDF is a function that provides information about how particles are distributed relative to one another. RMSD may be used to track changes in the conformation of molecules over time. Free energy calculations may help estimate the stability of different configurations and/or states, such as ligan binding to a receptor and/or the folding of a protein.

The Spike X Detect panel may be ideal for individuals experiencing unexplained symptoms after infection and/or vaccination and/or those with chronic health concerns. Key applications may include post-infection and/or post-vaccination symptom management, autoimmune and/or chronic inflammation detection, neurological and/or cardiovascular health monitoring, personalized peptide treatment planning, identifying triggers for accelerated cancer signaling, and/or monitoring metabolic, protein, and/or immune function stability.

In continued reference to FIG. 1, in an embodiment, at least a processor 108 may be configured to receive transcriptomic data 124. “Transcriptomic data,” as used herein, refers to the information derived from the transcriptome. The transcriptome may reflect genes that are actively being expressed, providing a snapshot of cellular activity, gene regulation, and/or responses to environmental factors. For example, transcriptomic data 124 may include bulk RNA sequencing data that measures average gene expression across a mixed population of cells such as gene counts, TPM/FPKM and differential expression. Transcriptomic data 124 may include single-cell RNA sequence data that captures gene expression at the level of individual cells such as cell by gene matrices, clustering, and cell-type identification. Transcriptomic data 124 may include single nucleus RNA sequence data that may utilized isolated nuclei instead of whole cells. Transcriptomic data 124 may include spatial transcriptomics which measure gene expression in situ while preserving tissue architecture. Transcriptomic data 124 may include total RNA-sequence data which captures all RNA species including coding and non-coding segments such as rRNA, tRNA, LincRNA, snRNA and the like. Transcriptomic data 124 may include mRNA-seq (poly-A-selected) which is enriched for polyadenylated transcripts. Transcriptomic data 124 may include ribosome profiling including Ribo-seq which captures ribosome protected mRNA fragments. In an embodiment, transcriptomic data 124 may include RNA, such as mRNA and/or non-coding RNAs. Transcriptomic data 124 may be obtained through RNA sequencing, microarrays, nanostring, qPCT/RT-qPCR, targeted RNA-seq panels, and/or northern blotting. Transcriptomic data 124 may include microRNA, long non-coding RNA, circular RNA, small nuclear and nuclear RNA, long read RNA, single cell multiomics, degradome sequencing, RNA cleavage, and the like. Further, in some cases transcriptomic data 124 may be obtained from a database 120 configured to store RNA and/or DNA sequencing.

With continued reference to FIG. 1, database 120 may contain RNA Expression Variant Instability Surveillance (REViSS) data. REViSS data may combine transcriptomics, spike-related synthetic signatures, and oncogenic potential scoring. This may utilize hybrid intelligence (aHI) to quantify and map systemic molecular instabilities. This may be used to stratify clinical risk post-vaccine, post-COVID, or chronic immune syndromes. REViSS data may include a REViSS score which may quantify signal-based molecular aberrations in patient samples. A REViSS score may integrate unfavorable gene expression, synthetic RNA contamination, and oncogenic potential. A REViSS score may provide a personalized molecular disruption index. REViSS data may be utilized to identify aberrations in immune regulation including cytokine signaling, antigen presentation, and TLR balance. REViSS data may identify core hallmark expression genes and proteins involved in homeostatic regulation. REViSS data may identify inflammatory resolution proteins that may misfire, including NF-kB, STAT3, and IL6. REViSS data may identify structural signaling collagens and laminins which show downregulation or mistranslation such as fibrous clots and tissue strands. REViSS data may identify ribosomal instability which contributes to mistranslation and misfolding. REViSS data may identify dysregulation of TP53, BRCA ½, RB1 and other key regulators. REViSS data may identify activation of proliferation and migration such as MYC, KRAS, and BCL2. REViSS data may identify oncogenic role-switching due to mistranslation. REViSS data may identify spike-linked synthetic elements and contaminants. REViSS data may aid in identifying signal disruption which precedes symptomatic disease. REViSS data may aid in identifying immune deregulation and oncogene instability which initiate chronic disease cascades. REViSS data may aid in the generation of precision and personalized peptides which offer selective and restorative correction. REViSS data may aid in designing personalized peptides to correct faulty molecular singles and direct spike mitigation. REViSS data may use hybrid intelligence (aHI) and omics data 116 for real-time surveillance. REViSS data may target immune recalibration and oncogene suppression and restore immune, transcriptional, and mitochondrial balance. This may aid in mapping molecular disorders in real time and enable signal-specific curated interventions. REViSS data may include genomic instability multiplier data (GIM). “Genomic instability multiplier data” as used in this disclosure is a quantitative surveillance construct that measures how multiple destabilizing molecular signals interact to amply genomic and transcriptomic instability beyond any single abnormality alone. GIM models the non-linear compounding effect of concurrent disruptions across DNA integrity, RNA fidelity, chromatin regulation, mitochondrial signaling, and immune stress pathways to produce a multiplier effect on disease risk, progression, and therapeutic resistance. GIM may include components such as but not limited to:
DNA-level instability: strand breaks, replication stress, CNVs, translocations
RNA instability: aberrant splicing, chimeric transcripts, frameshifts, persistence of foreign or dysregulated RNA species
Epigenetic dysregulation: chromatin accessibility shifts, methylation errors, histone imbalance
Mitochondrial-genomic crosstalk failure: mtDNA stress signaling, 12S/16S rRNA dysregulation, ROS amplification
Immune-inflammatory pressure: interferon exhaustion, chronic innate activation, checkpoint distortion
   Each domain alone may be clinically silent or subthreshold. When combined, they multiply system instability accelerating: oncogenic transformation; neurodegeneration; immune collapse or autoimmunity; and treatment failure and relapse. GIM functions as a derived index layered on top of RNA-seq and multi-omic inputs, converting distributed molecular noise into a single instability acceleration score used for: early risk stratification, longitudinal surveillance, therapy timing and personalization; and prediction of rapid phase transitions (e.g. stable→unstable states). GIM aids in quantifying how close a system is to losing control. REViSS data may be used along with transcriptomic data 124 to generate a composition of a personalized spike neutralization multivalent peptide sequence.

Still referring to FIG. 1, in an embodiment, at least a processor 108 may be configured to analyze transcriptomic data 124 using bioinformatic surveillance, modeling, ranking, mapping, and confidence selection. “Bioinformatic surveillance,” as used throughout this disclosure, refers to the ongoing monitoring and analysis of transcriptomic data 124 using computational tools designed to detect relevant biological patterns, anomalies, and trends. Bioinformatic surveillance may allow for the dynamic tracking of gene expression across different conditions and/or over time. Further, bioinformatic surveillance may identify unusual patterns in data, such as aberrant gene expression linked to disease states, and/or for monitoring changes in gene activity in response to treatments and/or environmental changes. In an embodiment, bioinformatic surveillance may include filtering raw data to remove noise, identifying outliers, and establishing baseline expression profiles. In some cases, bioinformatic surveillance may include visual tools such as heatmaps, and/or volcano plots to track expression levels across multiple samples and/or conditions.

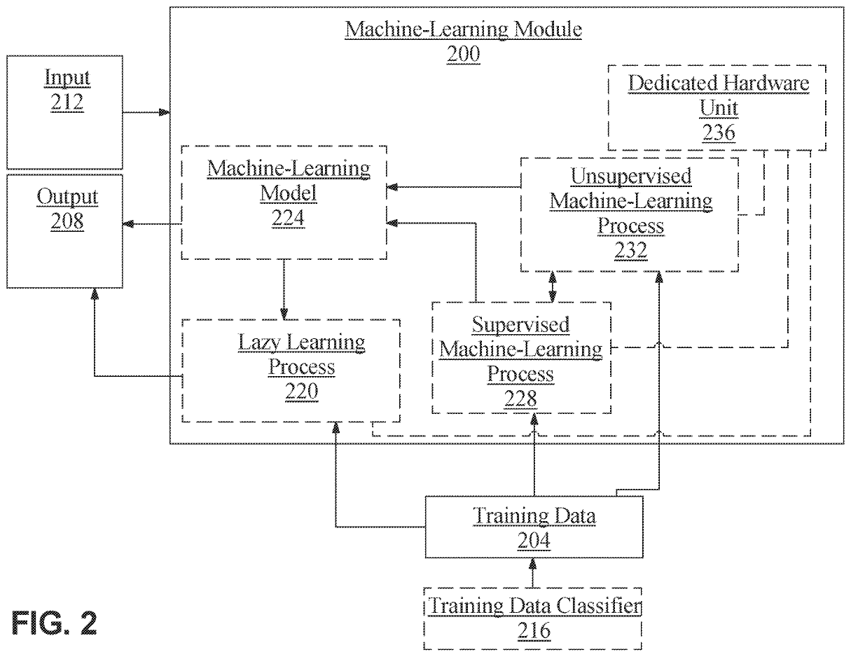

In continued reference to FIG. 1, as used herein, “modeling” refers to the construction of computational models that describe the relationships between different variables in transcriptomic data 124. This may involve the development of statistical, machine-learning, and/or systems biology models to predict gene expression behavior and/or interactions. The goal of modeling is to use mathematical and/or computational representations to simulate how genes and their regulatory elements interact under specific biological conditions. For example, and without limitation, a predictive model may be used to assess how gene expression changes with variations in environmental conditions, and/or to predict the effects of genetic mutations. In an embodiment, differential expression analysis, including algorithms such as DESeq2, edgeR, and/or limma may be used to identify genes that are significantly upregulated and/or downregulated across experimental conditions. The predictive model may identify gene expression changes correlated to variations in environmental conditions. The predictive model may identify predicted gene expression changes correlated to variations in environmental conditions. The predictive model may be implemented as any machine-learning model 224 as described below in more detail in reference to FIG. 2. “Predicted gene expression changes” as used in this disclosure, includes any expected alteration in a gene or its product based on a computational or statistical analysis. A predicted gene expression change may include an inferred effect on a DNA sequence, an RNA transcript, and/or a protein encoded by the gene. Examples of predicted gene expression change include but are not limited to a predicted amino acid change, a predicted loss of function, a predicted splice alteration, a predicted regulatory change, a predicted structural change and the like. Further, in some embodiments, gene co-expression networks may be constructed based on correlated gene expression patterns to uncover modules of co-expressed genes that may share regulatory mechanisms. In some cases, machine-learning algorithms such as clustering, decision trees, and/or deep learning may be trained on transcriptomic data 124 to predict biological outcomes, such as the likelihood of disease progression.

Continuing to reference FIG. 1, as used throughout this disclosure, “ranking” refers to the process of prioritizing or scoring genes, pathways, or features based on their relevance to the research question or based on statistical metrics such as fold-change, p-value, or significance. In an embodiment, ranking may aid researchers in focusing on the most significant genes and/or molecular features that are most likely to contribute to the biological processes under study, whether that's a disease mechanism, a therapeutic response, and/or a genetic variation. In an embodiment, ranking may be used in parallel with one or more processes as discussed throughout this disclosure. In some cases, ranking may be accomplished using gene prioritization, which may use metrics such as fold-change, p-values, and/or false discovery rates to rank genes in terms of their importance and/or relevance. Alternatively, ranking may be accomplished using pathway analysis, wherein entire biological pathways are ranked based on the collective expression patterns of genes involved in those pathways. Pathways may be ranked by how significantly their gene expression is altered across conditions, such as in response to a drug treatment and/or disease state.

In further reference to FIG. 1, “mapping,” as used herein, refers to the process of linking transcriptomic data 124 to known biological structures or pathways. In an embodiment, mapping may utilize one or more external databases 120 and/or reference genome annotations. The goal of mapping is to assign the gene expression data to specific biological contexts to interpret the biological significance of the data. Mapping may allow researchers to visualize how specific genes contribute to cellular processes and/or how changes in gene expression may correlate with disease mechanisms. In an embodiment, mapping may include genet ontology (GO) mapping, pathway mapping, and/or chromosomal mapping. GO mapping may include mapping differentially expressed genes to GO terms, which may aid in categorizing them based on their biological function, cellular localization, and/or molecular activity. Pathway mapping may include integrating transcriptomic data 124 with pathway databases, such as KEGG, Reactome, and/or Ingenuity Pathway Analysis (IPA) to determine how changes in gene expression may impact specific biological pathways. Chromosomal mapping may include associating gene expression data with specific chromosomal locations and/or genomic regions, such as identifying genes located near known mutations and/or in regions linked to disease susceptibility.

With continued reference to FIG. 1, “confidence selection,” as used throughout this disclosure, refers to assessing and selecting results from the analysis based on their statistical significance and reliability. This may ensure that the conclusions drawn from transcriptomic data 124 are robust and not due to random noise and/or errors in the data. In an embodiment, confidence selection aids in refining the results by filtering out low-confidence data and focusing on eth most reliable findings. Further, confidence selection may ensure that conclusions about gene expression, pathway activation, and/or disease association are statistically meaningful. Methods for confidence selection may include statistical confidence, bootstrap methods, and/or cross-validation. Still referring to FIG. 1, in an embodiment, at least a processor 108 is configured to output a personalized spike neutralization multivalent peptide sequence as a function of the transcriptomic data 124. In addition to genomic data, profiling the immune response (e.g. by analyzing HLA (Human Leukocyte Antigen) types, which influence immune recognition) may be used to predict which peptide sequences are more likely to activate an individual's immune system effectively. In an embodiment, epitope mapping may be used to generate peptides that correspond to critical regions of the spike protein. An “epitope map” as used in this disclosure, is a representation of the locations on an antigen where antibodies bind. An epitope map may aid in identifying which specific amino acids or regions of an antigen are recognized by an antibody; how antibodies interact with the antigen's structure; which epitopes are immunodominant and the like. An epitope map may be generated using shotgun mutagenesis to mutate residues to identify what changes disrupt antibody binding. An epitope map may be generated using peptide scanning to test overlapping peptides to find linear epitopes. An epitope map may be generated using structural biology to visualize antibody-antigen complexes at high resolution. An epitope map may be generated using a computational prediction to infer epitopes from sequences or structures. An epitope map may be helpful to understand antibody mechanisms of action, guide personalized spike neutralization multivalent peptide sequence 136 design, support therapeutic antibody development, and improve diagnostics. These peptides may be designed to bind specifically to the spike protein, preventing it from interacting with human cell receptors, thereby neutralizing the virus. In an embodiment, a confidence selection may indicate a threshold specifying a specific value or range that defines an acceptable performance and conclusion dawn from transcriptomic data 124. For instance and without limitation, the threshold may be anywhere above 50% indicates an acceptable performance and conclusion drawn from transcriptomic data 124. In yet another non-limiting example, the threshold may be anywhere above 75% indicates an acceptable performance and conclusion drawn from transcriptomic data 124. In yet another non-limiting example, the threshold may be a range where a confidence selection between 51-100% indicates an acceptable performance and conclusion drawn from transcriptomic data 124.

In continued reference to FIG. 1, in an embodiment, at least a processor 108 may be further configured to analyze transposable element activity. Wherein at least a processor 108 may identify activation and/or suppression of transposable elements, such as LINE-1 retrotransposons, and predict potential genomic disruptions and/or retro-integration. For purposes of this disclosure, “transposable element activity” refers to the process by which transposable elements (TEs), also known as ‘jumping genes,’ move or copy themselves from one location in the genome to another. “TEs,” as used herein, are DNA sequences that can change their position within the genome. TEs may lead to genetic variation and/or influence the expression of nearby genes. In an embodiment, TEs may be classified into two major types: Class I (retrotransposons), which may move using an RNA intermediate, and Class II (DNA transposons), which may move directly as DNA. As used herein, “LINE-1 retrotransposons” are a type of retrotransposon. Further, “retrotransposon,” for purposes of this disclosure, are a class of transposable elements that can move within the genome using an RNA intermediate.

Further referencing FIG. 1, for purposes of this disclosure, “genetic disruptions” refer to alterations or disturbances in the normal structure, function, or regulation of genes and genomic sequences. In an embodiment, genetic disruptions may lead to changes in cellular behavior, gene expression, and/or overall organismal function. Genetic disruptions may result from a variety of factors, including, but not limited to mutations, deletions, insertions, rearrangements, and/or changes in regulatory mechanisms. In an embodiment, gene disruptions may contribute to disease processes, including genetic disorders, cancer, and/or developmental abnormalities. “Retro-integration,” as used herein, is the process by which RNA is reverse-transcribed into DNA and integrated into the genome. In an embodiment, retro-integration is a type of retrotransposon wherein genetic material moves within the genome through an RNA intermediate. Retro-integration may be associated with the activity of retrotransposons, such as LINE-2, and/or retroviruses. In some cases, retro-integration may lead to genomic instability, mutation, and/or changes in gene expression.

In an embodiment, at least a processor 108 may detect reverse-loop embedding events of mRNA sequences, wherein at least a processor 108 may use a combination of transcriptomic data 124 and molecular simulations 132 to predict the likelihood of retro-integration into the human genome. In an embodiment, reverse embedding events of mRNA sequences may be analyzed for their impact on gene regulation using transcriptomic data 124 and genomic data. For purposes of this disclosure, “reverse-loop embedding events” refer to a hypothetical or specialized process in which RNA undergoes an unusual or non-canonical interaction with the host genome. In many cases, the RNA may be from an external source, such as mRNA vaccine, and/or viral RNA. Specifically, reverse-loop embedding events may involve the integration of RNA into the genome in a manner wherein the RNA forms a looped structure that facilitates its reverse transcription and subsequent embedding into the DNA. As used herein, “retro-integration” refers to the process by which RNA is reverse-transcribed into DNA and subsequently integrated in the host genome. This process is a form of retrotransposon and may involve the reverse flow of genetic information from RNA to DNA, in contrast to the usual transcription process wherein DNA is transcribed into RNA.

In further reference to FIG. 1, in an embodiment, at least a processor 108 may be further configured to instantiate one or more ribosomal frameshifting detection algorithms, wherein at least a processor 108 analyzes ribosomal translation fidelity and identifies potential frameshifting events linked to spike protein expression using the one or more ribosomal frameshifting detection algorithms. In such an embodiment, at least a processor 108 may identify programmed ribosomal frameshifting events triggered by viral RNA and/or synthetic mRNA, using bioinformatic algorithms to assess translation fidelity and/or downstream effects on protein functionality. “Frameshifting,” as used herein, is a genetic phenomenon where the ribosome misreads the mRNA sequence, altering the reading frame and producing incorrect protein sequences. In an embodiment, frameshifting events may lead to dysfunctional proteins, genomic instability, and/or disease. In some cases, such as with viral infections, frameshifting events may include a programmed event that serves to produce multiple proteins from a single RNA sequence. For purposes of this disclosure, “ribosomal translation fidelity” refers to the accuracy with which the ribosome synthesizes proteins by translating the messenger RNA (mRNA) into a corresponding amino acid sequence during protein synthesis. In an embodiment, ribosomal translation fidelity may be a measure of the accuracy of incorporation of the correct amino acids into a growing polypeptide chain in accordance with the genetic code encoded in the mRNA. High translation fidelity may be crucial for maintaining the integrity of cellular processes and/or avoiding the production of faulty proteins, which may lead to dysfunctional cellular activities and/or disease.

Continuing to reference FIG. 1, in an embodiment, at least a processor 108 may be further configured to analyze non-Mendelian strand mRNA (nms-mRNA) integration, wherein at least a processor 108 may detect integration of nms-mRNA into the human transcriptome and map its functional impact on gene expression. In such an embodiment, at least a processor 108 may utilize high-throughput sequencing, mapping algorithms, and/or functional impact modeling. As used herein, “nms-mRNA” refers to unconventional or atypical patterns of gene expression and RNA behavior that do not strictly follow the classical Mendelian inheritance principles. In an embodiment, non-Mendelian inheritance mechanisms may include mitochondrial inheritance, genomic imprinting, epigenetic modifications, and/or nuclear-cytoplasmic interactions. In some embodiments, nms-mRNA may involve asymmetric transcription and/or strand-specific RNA synthesis, which may result in non-classical mRNA expression patterns.

With further reference to FIG. 1, in an embodiment, at least a processor 108 may be further configured to identify and mitigate immune system hyperactivation, wherein at least a processor 108 may integrate cytokine profiling and immune cell phenotyping to assess the need for immune modulation post-vaccination. In such an embodiment, at least a processor 108 may profile immune responses, including cytokine signaling and immune cell phenotyping, to recalibrate adaptive and innate immune functions following spike protein exposure. For purposes of this disclosure, “immune system hyperactivation” refers to an exaggerated or excessive activation of the immune system beyond the normal response to infection, injury, or other immune challenges. A state of hyperactivation may lead to the immune system attacking not only harmful pathogens, such as bacteria, viruses, and/or fungi, but may also lead to the immune system attacking the body's own cells and tissues, resulting in inflammation and/or potential tissue damage. “Cytokine integration,” as used throughout this disclosure, refers to the process by which cytokines interact with other signaling pathways and systems within the body to regulate immune responses, inflammation, and cellular activities. As used herein, “cytokines” are signaling molecules of the immune system. For purposes of this disclosure, “phenotyping” is the process of observing, measuring, and analyzing the physical and biological characteristics of an organism. Biological characteristics, otherwise described as phenotypes, may be a result of the interaction between genotype, or genetic makeup, and the environment.

In continued reference to FIG. 1, at least a processor may be further configured to detect oxidative stress, unfolded protein response (UPR), and/or endoplasmic reticulum (ER) stress, using methods for modulating repair pathways to restore cellular homeostasis. As used herein, “oxidative stress” refers to an imbalance between the production of reactive oxygen species (ROS) and the body's ability to neutralize or detoxify them using its antioxidant defenses. ROS may include highly reactive molecules that contain oxygen, such as free radicals and/or non-radical molecules. Under normal conditions, ROS are produced as byproducts of cellular metabolism, particularly during mitochondrial respiration, but they are generally neutralized by antioxidants, such as glutathione, superoxide dismutase, and/or catalase. For purposes of this disclosure, “ER stress” refers to a condition in which the ER experiences an imbalance between the demand for protein folding and its capacity to properly fold proteins. ER stress may occur when the ER is overwhelmed with improperly folded and/or unfolded proteins, leading to dysfunction in its normal processes. This phenomenon may trigger a cellular response known as the unfolded protein response (UPR), aimed at restoring homeostasis within the ER.

With continued reference to FIG. 1, in an embodiment, at least a processor 108 may be further configured to instantiate an algorithm for variant-specific modeling of spike protein dynamics, wherein at least a processor 108 may be capable of adapting peptide sequences to neutralize variant-specific spike proteins more effectively. In such an embodiment, the algorithm for variant-specific modeling of spike protein dynamics may include a predictive model, which may evaluate variant-specific protein dynamics, immune evasion potential, and/or therapeutic efficacy of neutralizing peptides. For purposes of this disclosure, “immune evasion potential” refers to the ability of pathogens or tumor cells to escape detection, attack, or elimination by the host's immune system. In one or more embodiments, the predictive model may include any machine-learning embodiment as discussed throughout this disclosure.

In further reference to FIG. 1, in an embodiment, at least a processor 108 may be further configured to monitor molecular and clinical markers post-treatment on a longitudinal scale. Further, such an embodiment may incorporate machine-learning models that may predict therapeutic outcomes. Machine-learning models may include any machine-learning as described throughout this disclosure.

With continued reference to FIG. 1, in an embodiment, at least a processor 108 may be further configured to implement a framework for ensuring compliance with bioethical standards and/or regulatory requirements, including preclinical validation protocols and/or transparent reporting mechanisms.

In an embodiment, the present disclosed systems and methods may produce a composition 136 of a post-vaccine spike neutralization multivalent peptide sequence, wherein the composition 136 of post-vaccine spike neutralization multivalent peptide sequence comprises anti-spike peptide, NLRP3 mitigation, CD40 mitigation, IL-mitigation, IL-10 potentiation, anti-galectin-3, KL-FG_RXKF, and EP-PEQL.

In an embodiment, the composition 136 may address the side effects of RNA-based COVID-19 vaccines, such as Pfizer/BioNTech's BNT162b2, Moderna's mRNA-1273, and Novavax's NVX-COV2373. In an embodiment, composition 136 may include eight specialized peptides designed to mitigate the persistent impact of the Spike protein and associated inflammatory and cardiovascular complications. For example, and without limitation the specialized peptides may include Anti-Spike Peptide (ASP), NLRP3 Mitigation, CD40 Mitigation, IL-6 Mitigation, IL-10 Potentiation, Anti-Galectin-3, KL-FG_RXKF, and EP-PEQL. The formulation of composition 136 may also include NAD+ (25 mg/ml). NAD+ (Nicotinamide adenine dinucleotide) may improve cellular energy metabolism, particularly through its role in the mitochondrial electron transport chain, where it supports ATP production. Further NAD+ may also play a key role in DNA repair, reducing oxidative stress, and maintaining cellular health by activating sirtuins, proteins linked to longevity and reduced inflammation. In composition 136, NAD+ may enhance cellular energy recovery and resilience, further supporting the peptide's ability to mitigate inflammation and promote tissue repair.

ASP may neutralize the spike protein produced by RNA-based vaccines, which encode the SARS-CoV-2 spike protein to elicit an immune response. In some cases, spike protein may persist in circulation for extended periods, contributing to cardiovascular complications such as myocarditis. Interaction between the spike protein's receptor-binding domain (RBD) and ACE2 receptors may occur, however the RBD may also perform interactions with other receptors, such as neuropilin-1 and CD147, further contributing to side effects. These interactions may trigger a range of pro-inflammatory and pro-coagulant processes, leading to cellular dysfunction and vascular inflammation. The ASP peptide is designed to bind with high affinity to the RBD of the spike protein derived from the RNA-based vaccines, preventing its interaction with human receptors, thereby mitigating a broader spectrum of downstream inflammatory and thrombotic effects associated with prolonged spike protein circulation. ASP may be developed by harnessing the spike protein's known interactions with its receptors and performed mutations at key peptide-protein interfaces to enhance binding affinity and stability. Then PBIMA-AI platform may be applied to optimize the peptide's pharmacokinetics for increased efficacy and safety. PBIMA-AI (Precision-Based Immunomolecular Augmentation) platform for peptide synthesis is described by U.S. application Ser. No. 19/445,844 and entitled “PRECISION-BASED IMMUNO-MOLECULAR AUGMENTATION (PBIMA) COMPUTERIZED SYSTEM, METHOD, AND THERAPEUTIC VACCINE filed on Jan. 12, 2026, the entirety of which is incorporated herein by reference in its entirety.

The NLRP3 Mitigation Peptide may be designed to inhibit the activation of the NLRP3 inflammasome, a key component of the innate immune system that responds to danger signals. Activation of the NLRP3 inflammasome may lead to the production of pro-inflammatory cytokines IL-1β and IL-18, which may be associated with inflammation, immune dysregulation, and/or tissue damage. The Spike protein may activate the NLRP3 inflammasome, contributing to inflammatory responses post-vaccination. By specifically targeting NLRP3 and its downstream pathways, the NLRP3 mitigation peptide may reduce hyperinflammatory reactions, which may be linked to adverse effects such as cardiovascular disease and neuroinflammation. In an embodiment, developing the NLRP3 Mitigation Peptide, may be accomplished by modeling known protein-protein interactions within the NLRP3 inflammasome to construct the peptide template. Further, mutations may be performed at key peptide-protein interfaces to enhance binding affinity and stability. PBIMA-AI platform may then be applied to optimize the peptide's pharmacokinetics for increased efficacy and safety.

The CD40 Mitigation Peptide may target the CD40/CD40L signaling axis, which may play a pivotal role in immune responses, including T cell activation and cytokine production. Overactivation of CD40 signaling, particularly due to interactions with the Spike protein, may be linked to excessive immune responses and cytokine storms. The CD40 mitigation peptide may be designed to modulate CD40 activity, reducing the release of pro-inflammatory cytokines such as IL-6 and TNF-α, thereby mitigating the hyperinflammatory state observed in severe COVID-19 cases and vaccine recipients. Moreover, CD40 signaling may be involved in immunothrombosis, where coagulation and immune pathways intersect, contributing to thrombotic events. By regulating CD40 activity, the CD40 mitigation peptide may reduce inflammation and help prevent vaccine-related thrombotic complications. Developing the CD40-CD40L Peptide Mitigator, may be accomplished by modeling known interactions between CD40 and CD40L to construct a peptide template that interrupts their co-stimulatory signaling. Further, targeted mutations may be introduced at key peptide-protein interfaces to enhance both binding affinity and stability. PBIMA-AI platform may then be used to optimize the peptide's pharmacokinetics and improve overall safety.

The IL-6 Mitigation Peptide may be designed to suppress the interleukin-6 (IL-6) signaling, a pro-inflammatory cytokine that plays a crucial role in the immune response. Elevated levels of IL-6 may be linked to cytokine storms, which contribute to systemic inflammation, tissue damage, and severe complications such as myocarditis and thrombosis. In the context of RNA-based COVID-19 vaccines, IL-6 levels can rise significantly, promoting an exaggerated immune response. The IL-6 mitigation peptide may help to modulate IL-6 production, preventing its pathological overexpression and reducing the risk of inflammatory side effects. Developing the IL-6 Mitigation Peptide, may be accomplished by modeling the interaction between IL-6 and its receptor complex, focusing on the key regions involved in cytokine signaling. Further, targeted mutations may introduced at key peptide-protein interfaces to enhance binding affinity and stability. PBIMA-AI platform may be applied to optimize the peptide's pharmacokinetics and improve overall safety.

The IL-10 Potentiation Peptide may be designed to induce the release of IL-10, an anti-inflammatory cytokine critical for regulating immune responses and preventing excessive inflammation. By enhancing IL-10 activity, this peptide may amplify its anti-inflammatory effects to counterbalance the heightened inflammatory responses triggered by the Spike protein and mRNA vaccines. Through increased IL-10 signaling, IL-10 potentiation peptide may help restore immune homeostasis, reduce inflammation, and protect against tissue damage caused by overactive immune responses. This potentiation may be particularly beneficial in preventing cytokine storms and lowering the risk of chronic inflammation following vaccination. In an embodiment, designing the IL-10 Potentiation Peptide may be accomplished by employing one or more machine learning algorithms to predict peptides capable of inducing IL-10. The one or more machine-learning algorithms 128 may identify T-helper epitopes that stimulate IL-10 production. PBIMA-AI platform may then be applied to optimize the peptide's pharmacokinetics, ensuring enhanced stability. A “machine-learning algorithm” as used in this disclosure, is a mathematical process that enables a computing device 104 to learn patterns from data and use those learned patterns to make predictions or decisions without being explicitly programmed to do so. A machine-learning algorithm 128 any include anything suitable for use as machine-learning model 224 as described below in more detail in reference to FIG. 2. A machine-learning algorithm 128 may be trained with training data, including any training data 204 as described in more detail below in reference to FIG. 2. In an embodiment, transcriptomic data 124 and/or REViSS data may be input into a machine-learning algorithm 128 to output the personalized spike neutralization multivalent peptide sequence composition 136. In an embodiment, the personalized spike neutralization multivalent peptide sequence composition 136 may be configured to minimize a side effect from a vaccination. This may include configuring the personalized spike neutralization multivalent peptide sequence composition 136 to minimize side effects such as pain, fever, chills, fatigue, headache, muscle or joint ache, exercise intolerance, brain fog, dizziness, sleep disturbances and the like. In such an instance, the machine-learning algorithm 128 may be trained with training data which contains a plurality of data inputs containing transcriptomic data 124 and REViSS data correlated to a plurality of outputs containing personalized spike neutralization multivalent peptide sequence compositions 136. The machine-learning algorithm 128 may be trained with training data utilizing any methodology as described below in more detail in reference to FIG. 2.

The Anti-Galectin-3 Peptide may target galectin-3, a multifunctional protein implicated in a wide range of cellular processes, including inflammation, fibrosis, and immune regulation. Galectin-3 may be upregulated in the lungs of COVID-19 patients and may be associated with chronic inflammatory conditions and fibrosis. Galectin-3's role in promoting fibrosis and inflammation may make it a key target for reducing long-term tissue damage, particularly in the cardiovascular and pulmonary systems. Galectin-3 may also contribute to myocardial dysfunction and heart failure. By blocking galectin-3 activity, Galectin-3 aims to reduce inflammation and fibrosis, which may improve overall patient outcomes post-vaccination. Developing the Anti-Galectin-3 Peptide may be accomplished by modeling the interactions between Galectin-3 and its binding proteins, introducing targeted mutations to enhance binding affinity and stability. PBIMA-AI platform may then be applied to optimize the peptide's pharmacokinetics.

The KL-FG_RXKF peptide may be designed to harness the protective effects of the Klotho protein, which is known for its antioxidant and anti-aging properties. Klotho may play a key role in mitigating oxidative stress, particularly in mitochondrial function, where reactive oxygen species (ROS) can lead to cellular damage and inflammation. By targeting mitochondrial pathways, KL-FG_RXKF peptide may reduce oxidative damage and help restore proper mitochondrial function, thereby improving cellular health and reducing inflammation. Klotho may be associated with the protection of cardiovascular and renal systems, making this peptide an important component in addressing vaccine-induced oxidative stress and related complications.

EP-PEQL is an engineered version of the ARA-290 peptide, known for its tissue-protective and anti-inflammatory properties. ARA-290 may be derived from erythropoietin and may reduce inflammation and promote tissue repair without stimulating erythropoiesis. EP-PEQL may target inflammatory pathways and supports cellular repair, particularly in the context of immune regulation. EP-PEQL may be designed to alleviate the inflammatory side effects of RNA-based vaccines by promoting tissue regeneration and modulating the immune response. Its neuroprotective effects may be particularly relevant in preventing neurological side effects post-vaccination.

TABLE 1
Unmodified Peptide Sequences:

| | Peptide | Sequence |
|---|---|---|
| Seq-1 | Anti-Spike Peptide | TGLDKGNHEGEDLGYQ |
| Seq-2 | NLRP3 Mitigation | YGRKKRRQRRRVELKKFKL |
| | | KLLLSV |
| Seq-3 | CD40 Mitigation | KLGLKGYY |
| Seq-4 | IL-6 Mitigation | NSDVDARRLLEATL |
| Seq-5 | IL-10 Potentiation | RPFERDISNVPFS |
| Seq-6 | Anti-Galectin-3 | IPGPTFLDPH |
| Seq-7 | KL-FG_RXKF | FQGTFPDGFLWAVGSAAYQ |
| | | TEGGWQQHGKG |
| Seq-8 | EP-PEQL | PEQLERALNSS |

The peptide sequences disclosed herein and listed in Table 1 correspond to unmodified peptides and are provided in the accompanying sequence listing, entitled “35877-100401_SL”. In certain embodiments, one or more of the disclosed peptides may be chemically modified. Such modifications may include, but are not limited to, N-terminal or C-terminal modifications, terminal amidation, acetylation, alkylation, PEGylation (e.g., attaching PEG4), conjugation to amino-acid-based or non-amino-acid-based linkers or spacers (e.g., GGL motifs, aminohexanoic acid (ahx), etc.), incorporation of substituted amino acids (e.g., 2,6-dimethy-substituted residues), and combinations thereof. In some embodiments, an amino-acid-based spacer or linker may serve as a potential binding site as shown for example in Table 2.

TABLE 2
Exemplary Modified Peptide Sequences.

| | Peptide | Sequence |
|---|---|---|
| Seq-1 | Anti-Spike Peptide | PEG4-GGL-TGLDKGNHEGEDLGYQ |
| | | -NH2 |
| Seq-2 | NLRP3 Mitigation | PEG4-GGL-YGRKKRRQRRR-ahx- |
| | | VELKKFKLKLLLSV-NH2 |
| Seq-3 | CD40 Mitigation | PEG4-GGL-KLGLKGYY-ahx-RGD |
| | | -NH2 |
| Seq-4 | IL-6 Mitigation | PEG4-GGL-NSDVDARRLLEATL- |
| | | NH2 |
| Seq-5 | IL-10 | PEG4-GGL-RPFERDISNVPFS- |
| | Potentiation | NH2 |
| Seq-6 | Anti-Galectin-3 | PEG4-GGL-IPGPTFLDPH-ahx-R |
| | | GD-NH2 |
| Seq-7 | KL-FG_RXKF | PEG4-GGL-FQGTFPDGFLWAVGSA |
| | | AYQTEGGWQQHGKG-ahx-RX- |
| | | (2,6-diMe)-KF-NH2 |
| Seq-8 | EP-PEQL | PEG4-GGL-PEQLERALNSS-NH2 |

Referring now to FIG. 2, an exemplary embodiment of a machine-learning module 200 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 204 to generate an algorithm instantiated in hardware or software logic, data structures, and/or functions that will be performed by a computing device/module to produce outputs 208 given data provided as inputs 212; this is in contrast to a non-machine learning software program where the commands to be executed are determined in advance by a user and written in a programming language.

Still referring to FIG. 2, “training data,” as used herein, is data containing correlations that a machine-learning process may use to model relationships between two or more categories of data elements. For instance, and without limitation, training data 204 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 2, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail below. Training data 204 used by machine-learning module 200 may correlate any input data as described in this disclosure to any output data as described in this disclosure. As a non-limiting illustrative example inputs may include epidemiological and/or genomic data, such as transcriptomic data and outputs may include observed patterns and predictions of the data.

Further referring to FIG. 2, training data may be filtered, sorted, and/or selected using one or more supervised and/or unsupervised machine-learning processes and/or models as described in further detail below; such models may include without limitation a training data classifier 216. Training data classifier 216 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 200 may generate a classifier using a classification algorithm, defined as a processes whereby a computing device and/or any module and/or component operating thereon derives a classifier from training data 204. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 216 may classify elements of training data to various sub-cohorts such as mutation versus normal.

Still referring to FIG. 2, a computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. A computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 2, a computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 2, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm:

$$l = \sqrt{{Σ}_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 2, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. A computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 2, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 2, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 2, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 2, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 200 pixels, however a desired number of pixels may be 228. Processor may interpolate the low pixel count image to convert the 200 pixels into 228 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 2, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 2, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 2, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset

$${X}_{max} : {X}_{n e w} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{n e w} = \frac{X - {X}_{m e a n}}{{X}_{max} - {X}_{min}} .$$

Feature scaling may include standardization, where a difference between X and Xₘₑₐₙ is divided by a standard deviation σ of a set or subset of values:

$${X}_{n e w} = \frac{X - {X}_{m e a n}}{σ} .$$

Scaling may be performed using a median value of a set or subset X_{median} and/or interquartile range (IQR), which represents the difference between the 25^{th} percentile value and the 50^{th} percentile value (or closest values thereto by a rounding protocol), such as:

$${X}_{n e w} = \frac{X - {X}_{median}}{IQR} .$$

Persons skilled in the art, upon reviewing the entirety of this disclosure, will be aware of various alternative or additional approaches that may be used for feature scaling.

Further referring to FIG. 2, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 2, machine-learning module 200 may be configured to perform a lazy-learning process 220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 2, machine-learning processes as described in this disclosure may be used to generate machine-learning models 224. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 2, machine-learning algorithms may include at least a supervised machine-learning process 228. At least a supervised machine-learning process 228, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include inputs as described above as inputs, outputs described above as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon reviewing the entirety of this disclosure, will be aware of various possible variations of at least a supervised machine-learning process 228 that may be used to determine relation between inputs and outputs. Supervised machine-learning processes may include classification algorithms as defined above.

With further reference to FIG. 2, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 2, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 2, machine learning processes may include at least an unsupervised machine-learning processes 232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 232 may not require a response variable; unsupervised processes 232 may be used to find interesting patterns and/or inferences between variables, to determine a degree of correlation between two or more variables, or the like.

Still referring to FIG. 2, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 2 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Still referring to FIG. 2, machine-learning module 200 may include a large language model (LLM). A “large language model,” as used herein, is a deep learning data structure that can recognize, summarize, translate, predict and/or generate text and other content based on knowledge gained from massive datasets. Large language models may be trained on large sets of data. Training sets may be drawn from diverse sets of data such as, as non-limiting examples, novels, blog posts, articles, emails, unstructured data, electronic records, and the like. In some embodiments, training sets may include a variety of subject matters, such as, as nonlimiting examples, medical report documents, electronic health records, entity documents, business documents, inventory documentation, emails, user communications, advertising documents, newspaper articles, and the like. In some embodiments, training sets of an LLM may include information from one or more public or private databases. As a non-limiting example, training sets may include databases associated with an entity. In some embodiments, training sets may include portions of documents associated with the electronic records correlated to examples of outputs. In an embodiment, an LLM may include one or more architectures based on capability requirements of an LLM. Exemplary architectures may include, without limitation, GPT (Generative Pretrained Transformer), BERT (Bidirectional Encoder Representations from Transformers), T5 (Text-To-Text Transfer Transformer), and the like. Architecture choice may depend on a needed capability such generative, contextual, or other specific capabilities.

With continued reference to FIG. 2, in some embodiments, an LLM may be generally trained. As used in this disclosure, a “generally trained” LLM is an LLM that is trained on a general training set comprising a variety of subject matters, data sets, and fields. In some embodiments, an LLM may be initially generally trained. Additionally, or alternatively, an LLM may be specifically trained. As used in this disclosure, a “specifically trained” LLM is an LLM that is trained on a specific training set, wherein the specific training set includes data including specific correlations for the LLM to learn. As a non-limiting example, an LLM may be generally trained on a general training set, then specifically trained on a specific training set. In an embodiment, specific training of an LLM may be performed using a supervised machine learning process. In some embodiments, generally training an LLM may be performed using an unsupervised machine learning process. As a non-limiting example, specific training set may include information from a database. As a non-limiting example, specific training set may include text related to the users such as user specific data for electronic records correlated to examples of outputs. In an embodiment, training one or more machine learning models may include setting the parameters of the one or more models (weights and biases) either randomly or using a pretrained model. Generally training one or more machine learning models on a large corpus of text data can provide a starting point for fine-tuning on a specific task. A model such as an LLM may learn by adjusting its parameters during the training process to minimize a defined loss function, which measures the difference between predicted outputs and ground truth. Once a model has been generally trained, the model may then be specifically trained to fine-tune the pretrained model on task-specific data to adapt it to the target task. Fine-tuning may involve training a model with task-specific training data, adjusting the model's weights to optimize performance for the particular task. In some cases, this may include optimizing the model's performance by fine-tuning hyperparameters such as learning rate, batch size, and regularization. Hyperparameter tuning may help in achieving the best performance and convergence during training. In an embodiment, fine-tuning a pretrained model such as an LLM may include fine-tuning the pretrained model using Low-Rank Adaptation (LoRA). As used in this disclosure, “Low-Rank Adaptation” is a training technique for large language models that modifies a subset of parameters in the model. Low-Rank Adaptation may be configured to make the training process more computationally efficient by avoiding a need to train an entire model from scratch. In an exemplary embodiment, a subset of parameters that are updated may include parameters that are associated with a specific task or domain.

With continued reference to FIG. 2, in some embodiments an LLM may include and/or be produced using Generative Pretrained Transformer (GPT), GPT-2, GPT-3, GPT-4, and the like. GPT, GPT-2, GPT-3, GPT-3.5, and GPT-4 are products of Open AI Inc., of San Francisco, CA. An LLM may include a text prediction based algorithm configured to receive an article and apply a probability distribution to the words already typed in a sentence to work out the most likely word to come next in augmented articles. For example, if some words that have already been typed are “Nice to meet”, then it may be highly likely that the word “you” will come next. An LLM may output such predictions by ranking words by likelihood or a prompt parameter. For the example given above, an LLM may score “you” as the most likely, “your” as the next most likely, “his” or “her” next, and the like. An LLM may include an encoder component and a decoder component.

Still referring to FIG. 2, an LLM may include a transformer architecture. In some embodiments, encoder component of an LLM may include transformer architecture. A “transformer architecture,” for the purposes of this disclosure is a neural network architecture that uses self-attention and positional encoding. Transformer architecture may be designed to process sequential input data, such as natural language, with applications towards tasks such as translation and text summarization. Transformer architecture may process the entire input all at once. “Positional encoding,” for the purposes of this disclosure, refers to a data processing technique that encodes the location or position of an entity in a sequence. In some embodiments, each position in the sequence may be assigned a unique representation. In some embodiments, positional encoding may include mapping each position in the sequence to a position vector. In some embodiments, trigonometric functions, such as sine and cosine, may be used to determine the values in the position vector. In some embodiments, position vectors for a plurality of positions in a sequence may be assembled into a position matrix, wherein each row of position matrix may represent a position in the sequence.

With continued reference to FIG. 2, an LLM and/or transformer architecture may include an attention mechanism. An “attention mechanism,” as used herein, is a part of a neural architecture that enables a system to dynamically quantify the relevant features of the input data. In the case of natural language processing, input data may be a sequence of textual elements. It may be applied directly to the raw input or to its higher-level representation.

With continued reference to FIG. 2, attention mechanism may represent an improvement over a limitation of an encoder-decoder model. An encoder-decider model encodes an input sequence to one fixed length vector from which the output is decoded at each time step. This issue may be seen as a problem when decoding long sequences because it may make it difficult for the neural network to cope with long sentences, such as those that are longer than the sentences in the training corpus. Applying an attention mechanism, an LLM may predict the next word by searching for a set of positions in a source sentence where the most relevant information is concentrated. An LLM may then predict the next word based on context vectors associated with these source positions and all the previously generated target words, such as textual data of a dictionary correlated to a prompt in a training data set. A “context vector,” as used herein, are fixed-length vector representations useful for document retrieval and word sense disambiguation.

Still referring to FIG. 2, attention mechanism may include, without limitation, generalized attention self-attention, multi-head attention, additive attention, global attention, and the like. In generalized attention, when a sequence of words or an image is fed to an LLM, it may verify each element of the input sequence and compare it against the output sequence. Each iteration may involve the mechanism's encoder capturing the input sequence and comparing it with each element of the decoder's sequence. From the comparison scores, the mechanism may then select the words or parts of the image that it needs to pay attention to. In self-attention, an LLM may pick up particular parts at different positions in the input sequence and over time compute an initial composition of the output sequence. In multi-head attention, an LLM may include a transformer model of an attention mechanism. Attention mechanisms, as described above, may provide context for any position in the input sequence. For example, if the input data is a natural language sentence, the transformer does not have to process one word at a time. In multi-head attention, computations by an LLM may be repeated over several iterations, each computation may form parallel layers known as attention heads. Each separate head may independently pass the input sequence and corresponding output sequence element through a separate head. A final attention score may be produced by combining attention scores at each head so that every nuance of the input sequence is taken into consideration. In additive attention (Bahdanau attention mechanism), an LLM may make use of attention alignment scores based on a number of factors. Alignment scores may be calculated at different points in a neural network, and/or at different stages represented by discrete neural networks. Source or input sequence words are correlated with target or output sequence words but not to an exact degree. This correlation may take into account all hidden states and the final alignment score is the summation of the matrix of alignment scores. In global attention (Luong mechanism), in situations where neural machine translations are required, an LLM may either attend to all source words or predict the target sentence, thereby attending to a smaller subset of words.

With continued reference to FIG. 2, multi-headed attention in encoder may apply a specific attention mechanism called self-attention. Self-attention allows models such as an LLM or components thereof to associate each word in the input, to other words. As a non-limiting example, an LLM may learn to associate the word “you”, with “how” and “are”. It's also possible that an LLM learns that words structured in this pattern are typically a question and to respond appropriately. In some embodiments, to achieve self-attention, input may be fed into three distinct fully connected neural network layers to create query, key, and value vectors. A query vector may include an entity's learned representation for comparison to determine attention score. A key vector may include an entity's learned representation for determining the entity's relevance and attention weight. A value vector may include data used to generate output representations. Query, key, and value vectors may be fed through a linear layer; then, the query and key vectors may be multiplied using dot product matrix multiplication in order to produce a score matrix. The score matrix may determine the amount of focus for a word should be put on other words (thus, each word may be a score that corresponds to other words in the time-step). The values in score matrix may be scaled down. As a non-limiting example, score matrix may be divided by the square root of the dimension of the query and key vectors. In some embodiments, the softmax of the scaled scores in score matrix may be taken. The output of this softmax function may be called the attention weights. Attention weights may be multiplied by your value vector to obtain an output vector. The output vector may then be fed through a final linear layer.

Still referencing FIG. 2, in order to use self-attention in a multi-headed attention computation, query, key, and value may be split into N vectors before applying self-attention. Each self-attention process may be called a “head.” Each head may produce an output vector and each output vector from each head may be concatenated into a single vector. This single vector may then be fed through the final linear layer discussed above. In theory, each head can learn something different from the input, therefore giving the encoder model more representation power.

With continued reference to FIG. 2, encoder of transformer may include a residual connection. Residual connection may include adding the output from multi-headed attention to the positional input embedding. In some embodiments, the output from residual connection may go through a layer normalization. In some embodiments, the normalized residual output may be projected through a pointwise feed-forward network for further processing. The pointwise feed-forward network may include a couple of linear layers with a ReLU activation in between. The output may then be added to the input of the pointwise feed-forward network and further normalized.

Continuing to refer to FIG. 2, transformer architecture may include a decoder. Decoder may a multi-headed attention layer, a pointwise feed-forward layer, one or more residual connections, and layer normalization (particularly after each sub-layer), as discussed in more detail above. In some embodiments, decoder may include two multi-headed attention layers. In some embodiments, decoder may be autoregressive. For the purposes of this disclosure, “autoregressive” means that the decoder takes in a list of previous outputs as inputs along with encoder outputs containing attention information from the input.

With further reference to FIG. 2, in some embodiments, input to decoder may go through an embedding layer and positional encoding layer in order to obtain positional embeddings. Decoder may include a first multi-headed attention layer, wherein the first multi-headed attention layer may receive positional embeddings.

With continued reference to FIG. 2, first multi-headed attention layer may be configured to not condition to future tokens. As a non-limiting example, when computing attention scores on the word “am,” decoder should not have access to the word “fine” in “I am fine,” because that word is a future word that was generated after. The word “am” should only have access to itself and the words before it. In some embodiments, this may be accomplished by implementing a look-ahead mask. Look ahead mask is a matrix of the same dimensions as the scaled attention score matrix that is filled with “0s” and negative infinities. For example, the top right triangle portion of look-ahead mask may be filled with negative infinities. Look-ahead mask may be added to scaled attention score matrix to obtain a masked score matrix. Masked score matrix may include scaled attention scores in the lower-left triangle of the matrix and negative infinities in the upper-right triangle of the matrix. Then, when the softmax of this matrix is taken, the negative infinities will be zeroed out; this leaves zero attention scores for “future tokens.”

Still referring to FIG. 2, second multi-headed attention layer may use encoder outputs as queries and keys and the outputs from the first multi-headed attention layer as values. This process matches the encoder's input to the decoder's input, allowing the decoder to decide which encoder input is relevant to put a focus on. The output from second multi-headed attention layer may be fed through a pointwise feedforward layer for further processing.

With continued reference to FIG. 2, the output of the pointwise feedforward layer may be fed through a final linear layer. This final linear layer may act as a classifier. This classifier may be as big as the number of classes that you have. For example, if you have 10,000 classes for 10,000 words, the output of that classifier will be of size 10,000. The output of this classifier may be fed into a softmax layer which may serve to produce probability scores between zero and one. The index may be taken of the highest probability score in order to determine a predicted word.

Still referring to FIG. 2, decoder may take this output and add it to the decoder inputs. Decoder may continue decoding until a token is predicted. Decoder may stop decoding once it predicts an end token.

Continuing to refer to FIG. 2, in some embodiment, decoder may be stacked N layers high, with each layer taking in inputs from the encoder and layers before it. Stacking layers may allow an LLM to learn to extract and focus on different combinations of attention from its attention heads.

With continued reference to FIG. 2, an LLM may receive an input. Input may include a string of one or more characters. Inputs may additionally include unstructured data. For example, input may include one or more words, a sentence, a paragraph, a thought, a query, and the like. A “query” for the purposes of the disclosure is a string of characters that poses a question. In some embodiments, input may be received from a user device. User device may be any computing device that is used by a user. As non-limiting examples, user device may include desktops, laptops, smartphones, tablets, and the like. In some embodiments, input may include any set of data associated with transcriptomic data.

With continued reference to FIG. 2, an LLM may generate at least one annotation as an output. At least one annotation may be any annotation as described herein. In some embodiments, an LLM may include multiple sets of transformer architecture as described above. Output may include a textual output. A “textual output,” for the purposes of this disclosure is an output comprising a string of one or more characters. Textual output may include, for example, a plurality of annotations for unstructured data. In some embodiments, textual output may include a phrase or sentence identifying the status of a user query. In some embodiments, textual output may include a sentence or plurality of sentences describing a response to a user query. As a non-limiting example, this may include restrictions, timing, advice, dangers, benefits, and the like.

Continuing to refer to FIG. 2, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 2, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 2, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 2, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 2, one or more processes or algorithms described above may be performed by at least a dedicated hardware unit 236. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 236 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 236 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 236 to perform one or more operations described herein, such as evaluation of model and/or algorithm outputs, one-time or iterative updates to parameters, coefficients, weights, and/or biases, and/or any other operations such as vector and/or matrix operations as described in this disclosure.

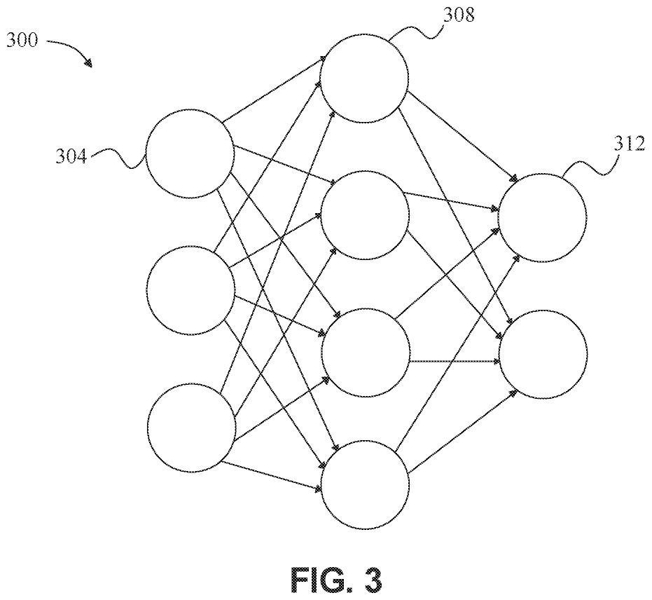

Referring now to FIG. 3, an exemplary embodiment of neural network 300 is illustrated. A neural network 300 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 304, one or more intermediate layers 308, and an output layer of nodes 312. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

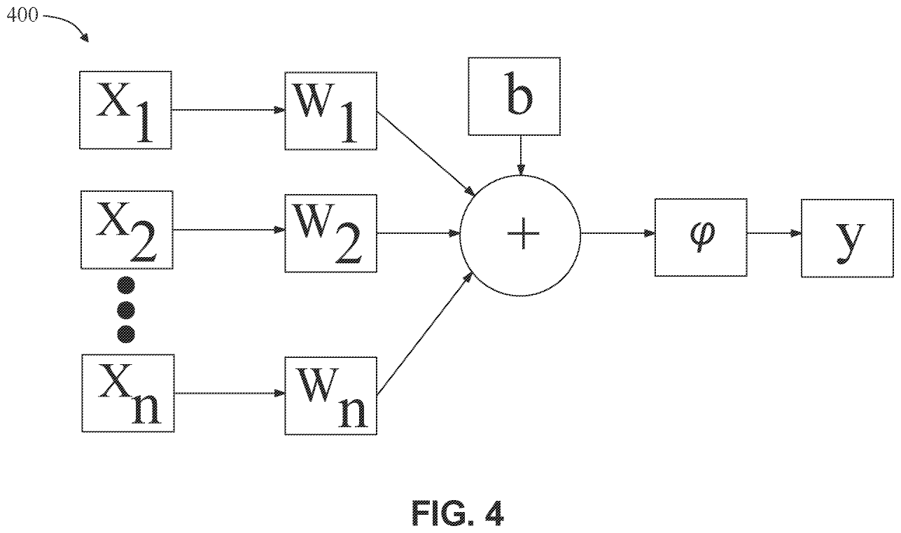

Referring now to FIG. 4, an exemplary embodiment of a node 400 of a neural network is illustrated. A node may include, without limitation a plurality of inputs xi that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as f(x)=tanh²(x), a rectified linear unit function such as f(x)=max(0, x), a “leaky” and/or “parametric” rectified linear unit function such as f(x)=max(ax, x) for some a, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as f(x)=x*sigmoid(x), a Gaussian error linear unit function such as f(x)=a(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xi that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

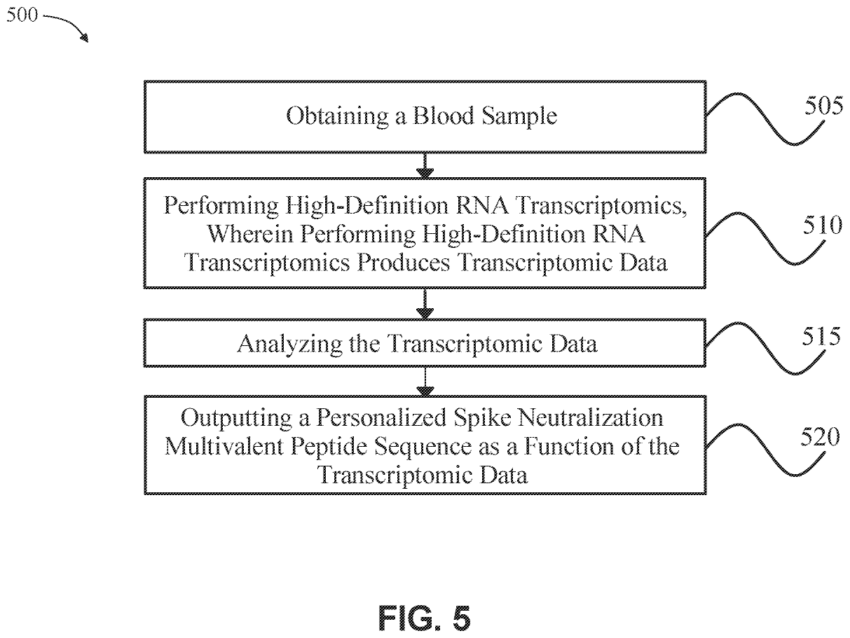

FIG. 5 illustrates an exemplary method 500 for post-vaccine spike neutralization multivalent peptide detection and engineering. Method 500 may include a step 505 of obtaining a blood sample. This may be implemented as described, without limitation, in reference to FIGS. 1-4. Method 500 may include a step 510 of performing high-definition RNA transcriptomics, wherein performing high-definition RNA transcriptomics produces transcriptomic data. This may be implemented, without limitation, as described in reference to FIGS. 1-4. Method 500 may include a step 515 of analyzing the transcriptomic data using bioinformatic surveillance, modeling, ranking, mapping, and confidence selection. In an embodiment, step 515 may further include utilizing one or more machine-learning algorithms, which may include an LLM. Further, in some cases, step 515 may further include using molecular simulation. This may be implemented, without limitation, as described in reference to FIGS. 1-4. Method 500 may include a step 520 of outputting a personalized spike neutralization multivalent peptide sequence as a function of the transcriptomic data. This may be implemented, without limitation, as described in reference to FIGS. 1-4

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

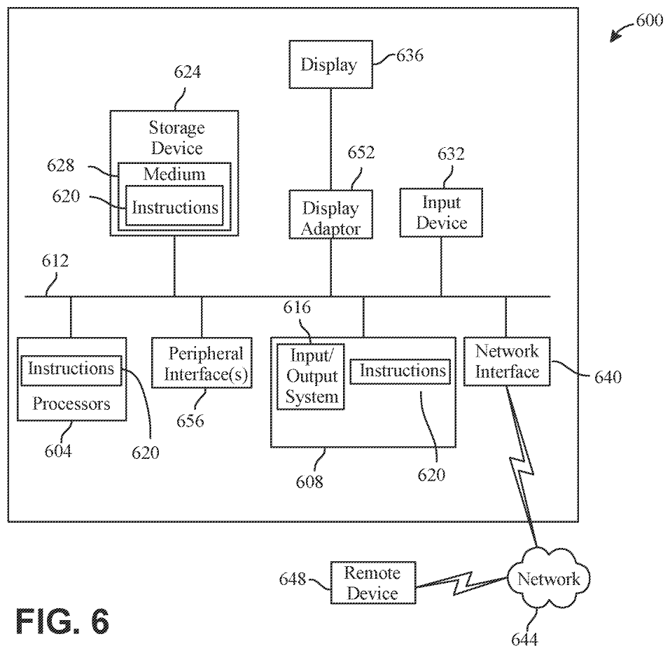

FIG. 6 shows a diagrammatic representation of one embodiment of a computing device in the exemplary form of a computer system 600 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 600 includes a processor 604 and a memory 608 that communicate with each other, and with other components, via a bus 612. Bus 612 may include any of several types of bus structures including, but not limited to, a memory bus, a memory controller, a peripheral bus, a local bus, and any combinations thereof, using any of a variety of bus architectures.

Processor 604 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 604 may be organized according to Von Neumann and/or Harvard architecture as a non-limiting example. Processor 604 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 608 may include various components (e.g., machine-readable media) including, but not limited to, a random-access memory component, a read only component, and any combinations thereof. In one example, a basic input/output system 616 (BIOS), including basic routines that help to transfer information between elements within computer system 600, such as during start-up, may be stored in memory 608. Memory 608 may also include (e.g., stored on one or more machine-readable media) instructions (e.g., software) 620 embodying any one or more of the aspects and/or methodologies of the present disclosure. In another example, memory 608 may further include any number of program modules including, but not limited to, an operating system, one or more application programs, other program modules, program data, and any combinations thereof.

Computer system 600 may also include a storage device 624. Examples of a storage device (e.g., storage device 624) include, but are not limited to, a hard disk drive, a magnetic disk drive, an optical disc drive in combination with an optical medium, a solid-state memory device, and any combinations thereof. Storage device 624 may be connected to bus 612 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 624 (or one or more components thereof) may be removably interfaced with computer system 600 (e.g., via an external port connector (not shown)). Particularly, storage device 624 and an associated machine-readable medium 628 may provide nonvolatile and/or volatile storage of machine-readable instructions, data structures, program modules, and/or other data for computer system 600. In one example, software 620 may reside, completely or partially, within machine-readable medium 628. In another example, software 620 may reside, completely or partially, within processor 604.

Computer system 600 may also include an input device 632. In one example, a user of computer system 600 may enter commands and/or other information into computer system 600 via input device 632. Examples of an input device 632 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 632 may be interfaced to bus 612 via any of a variety of interfaces (not shown) including, but not limited to, a serial interface, a parallel interface, a game port, a USB interface, a FIREWIRE interface, a direct interface to bus 612, and any combinations thereof. Input device 632 may include a touch screen interface that may be a part of or separate from display 636, discussed further below. Input device 632 may be utilized as a user selection device for selecting one or more graphical representations in a graphical interface as described above.

A user may also input commands and/or other information to computer system 600 via storage device 624 (e.g., a removable disk drive, a flash drive, etc.) and/or network interface device 640. A network interface device, such as network interface device 640, may be utilized for connecting computer system 600 to one or more of a variety of networks, such as network 644, and one or more remote devices 648 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 644, may employ a wired and/or a wireless mode of communication. In general, any network topology may be used. Information (e.g., data, software 620, etc.) may be communicated to and/or from computer system 600 via network interface device 640.

Computer system 600 may further include a video display adapter 652 for communicating a displayable image to a display device, such as display device 636. Examples of a display device include, but are not limited to, a liquid crystal display (LCD), a cathode ray tube (CRT), a plasma display, a light emitting diode (LED) display, and any combinations thereof. Display adapter 652 and display device 636 may be utilized in combination with processor 604 to provide graphical representations of aspects of the present disclosure. In addition to a display device, computer system 600 may include one or more other peripheral output devices including, but not limited to, an audio speaker, a printer, and any combinations thereof. Such peripheral output devices may be connected to bus 612 via a peripheral interface 656. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and compositions according to the present disclosure. Accordingly, this description is meant to be taken only by way of example, and not to otherwise limit the scope of this invention. Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for post-vaccine spike neutralization multivalent peptide detection and engineering, wherein the system comprises:
at least a processor; and
a memory communicatively connected to the at least a processor, wherein the memory contains instructions configuring the at least a processor to:
receive transcriptomic data;
analyze the transcriptomic data using bioinformatic surveillance, modeling, ranking, mapping, and confidence selection; and
output a personalized spike neutralization multivalent peptide sequence as a function of the transcriptomic data.

2. The system of claim 1, wherein analyzing the transcriptomic data further comprises utilizing one or more machine-learning algorithms.

3. The system of claim 2, wherein the machine-learning algorithm further comprises generating a predictive model wherein the predictive model identifies gene expression changes correlated to variations in environmental conditions.

4. The system of claim 3, wherein the predictive model identifies predicted gene expression changes correlated to variations in environmental conditions.

5. The system of claim 1, wherein analyzing the transcriptomic data further comprises utilizing a molecular simulation.

6. The system of claim 5, wherein the molecular simulation is selected from a group consisting of molecular dynamics (MD) simulations, Monte Carlo (MC) simulations, quantum mechanical simulations and course-grained simulations.

7. The system of claim 5, wherein the molecular simulation is configured to detect a pattern in transcriptomic data linked to one or more disease states.

8. The system of claim 1, wherein outputting the personalized spike neutralization multivalent peptide sequence further comprises generating an epitope map configured to output a peptide configured to bind to a spike protein and prevent interaction with a human cell receptor.

9. The system of claim 1, wherein the personalized spike neutralization multivalent peptide sequence is output and configured to minimize a side effect from a vaccination.

10. A method for post-vaccine spike neutralization multivalent peptide detection and engineering, wherein the method comprises:
obtaining a blood sample;
performing high-definition RNA transcriptomics, wherein performing high-definition RNA transcriptomics produces transcriptomic data;
analyzing the transcriptomic data using bioinformatic surveillance, modeling, ranking, mapping, and confidence selection; and
outputting a personalized spike neutralization multivalent peptide sequence as a function of the transcriptomic data.

11. The method of claim 10, wherein analyzing the transcriptomic data further comprises utilizing one or more machine-learning algorithms.

12. The method of claim 11, wherein the machine-learning algorithm further comprises generating a predictive model wherein the predictive model identifies gene expression changes correlated to variations in environmental conditions.

13. The method of claim 12, wherein the predictive model identifies predicted gene expression changes correlated to variations in environmental conditions.

14. The method of claim 10, wherein analyzing the transcriptomic data further comprises utilizing a molecular simulation.

15. The method of claim 14, wherein the molecular simulation is selected from a group consisting of molecular dynamics (MD) simulations, Monte Carlo (MC) simulations, quantum mechanical simulations and course-grained simulations.

16. The method of claim 14, wherein the molecular simulation is configured to detect a pattern in transcriptomic data linked to one or more disease states.

17. The method of claim 10, wherein outputting the personalized spike neutralization multivalent peptide sequence further comprises generating an epitope mapping configured to output a peptide configured to bind to a spike protein and prevent interaction with a human cell receptor.

18. The method of claim 10, wherein the personalized spike neutralization multivalent peptide sequence is output and configured to minimize a side effect from a vaccination.

19. A post-vaccine spike neutralization multivalent peptide sequence, wherein the post-vaccine spike neutralization multivalent peptide sequence comprises:
anti-spike peptide;
NLRP3 mitigation:
CD40 mitigation;
IL-mitigation;
IL-10 potentiation;
anti-galectin-3;
KL-FG_RXKF; and
EP-PEQL.

20. The post-vaccine spike neutralization multivalent peptide of claim 19, further comprising nicotinamide adenine dinucleotide.
